(19) United States
(12) Patent Application Publication
(10) Pub. No.: US 2008/0139514 A1
(43) Pub. Date: Jun. 12, 2008

(54) DIPHOSPHONIC ACID PHARMACEUTICAL COMPOSITIONS

(76) Inventors: Subhash Pandurang Gore, Sholapur (IN); Dokku Somasundara Rao, Ongole (IN); Indu Bhushan, Hyderabad (IN); Mailatur Sivaraman Mohan, Hyderabad (IN); Kolakaluri Uma Maheswara Prasad, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/945,507

(22) Filed: Nov. 27, 2007

Related U.S. Application Data
(60) Provisional application No. 60/893,148, filed on Mar. 6, 2007.

(30) Foreign Application Priority Data
Nov. 29, 2006 (IN) 2215/CHE/2006

(51) Int. Cl. A61K 31/663

(52) U.S. Cl. 514/108

(57) ABSTRACT

Solid pharmaceutical compositions comprising a diphosphonic acid or a pharmaceutically acceptable salt, solvate, hydrate, prodrug, enantiomer or racemic mixture thereof. The invention includes compositions with inner and outer phases, wherein the inner phase comprises no more than about 80% w/w and the outer phase comprises at least about 20% w/w.

### INTRODUCTION TO THE INVENTION

The present invention relates to pharmaceutical compositions comprising a diphosphonic acid or a pharmaceutically acceptable salt, solvate, hydrate, prodrug, enantiomer or racemic mixture thereof.

Ibandronic acid is a nitrogen-containing diphosphonic acid. Ibandronic acid has a chemical name 3-(N-methyl-N-pentyl) amino-1-hydroxypropane-1,1-diphosphonic acid, or [1-hydroxy-3-(methyl-pentyl-amino)-1-phosphono-propyl]phosphonic acid, with the molecular formula C₉H₂₃NO₇P₂ and a molecular weight of 319.229. Ibandronic acid is a white to off-white powder. It is freely soluble in water and practically insoluble in organic solvents. Ibandronic acid has the structural formula shown below.

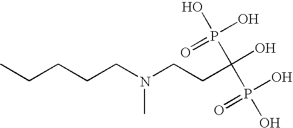

Pharmaceutically acceptable salts of biphosphonic acids include but are not limited to base salts of biphosphonic acid such as ammonium salts, alkali metal salts such as potassium and sodium (including mono-, di-, tri-sodium) salts, alkaline earth metal salts such as calcium and magnesium salts, salts with organic bases such as dicyclohexylamine salts, N-methyl-D-glucamine salts, and salts with amino acids such as arginine, lysine, etc.

Ibandronic acid inhibits osteoclast-mediated bone resorption. The action of ibandronic acid on bone tissue is based on its affinity for hydroxyapatite, which is part of the mineral matrix of bone. Ibandronic acid inhibits osteoclast activity and reduces bone resorption and turnover. In postmenopausal women, it reduces the elevated rate of bone turnover, leading to, on average, a net gain in bone mass. It may also be used to treat hypercalcemia (elevated blood calcium levels).

The sodium salt of ibandronic acid exists in two polymorphic forms wherein the two polymorphs have similar solubility profiles. The sodium salt is quite stable when stored at room temperature.

Ibandronic acid sodium salt is present in products marketed under the trade names BONIVA®, BONDRANAT® and BONVIVA®. BONIVA is available as a white, oblong, 2.5 mg film-coated tablet for daily oral administration or as a white, oblong, 150 mg film-coated tablet for once-monthly oral administration. One 2.5 mg film-coated tablet contains 2.813 mg ibandronate monosodium monohydrate, equivalent to 2.5 mg of ibandronic acid. One 150 mg film-coated tablet contains 168.75 mg of ibandronate monosodium monohydrate, equivalent to 150 mg of ibandronic acid. BONIVA also contains the following inactive ingredients: lactose monohydrate, povidone, microcrystalline cellulose, crospovidone, purified stearic acid, colloidal silicon dioxide, and purified water. The tablet film coating contains hypromellose, titanium dioxide, talc, polyethylene glycol 6000, and purified water.

BONIVA Injection is intended for intravenous administration only. BONIVA Injection is available as a sterile, clear, colorless, ready-to-use solution in a prefilled syringe that delivers 3.375 mg of ibandronate monosodium salt monohydrate in 3 mL of solution, equivalent to a dose of 3 mg of ibandronic acid. Inactive ingredients include sodium chloride, glacial acetic acid, sodium acetate and water.

Ibandronic acid derivatives are disclosed in U.S. Pat. No. 4,927,814. U.S. Pat. Nos. 6,294,196, 6,143,326, 6,623,755, 6,692,764, and 6,372,728 disclose pharmaceutical compositions of ibandronic acid or its salts. International Application Publication Nos. WO 2005/063218, WO 2005/030177, WO 01/32185, and WO 00/61111 disclose pharmaceutical compositions of ibandronic acid or its salts.

U.S. Patent Application Publication No. 2004/0121007 discloses oral formulations of ibandronic acid sodium salt and U.S. Patent Application Publication No. 2005/0089573 discloses oral ibandronate formulations. U.S. Patent Application Publication No. 2004/0147484 discloses compounds and compositions for the delivery of bisphosphonates. International Application Publication No. WO 2003/0181421 discloses a method of treatment using ibandronate.

Bisphosphonates are in a class of drugs, which are irritant to skin and mucous membranes, and when given orally on a continuous basis may result in digestive tract side effects such as esophageal adverse events or gastrointestinal disturbances. As a consequence, and due to their low oral bioavailability, the oral route of administration must, to date, follow inconvenient recommendations of use for the patient.

Hence there is a clear need for a treatment offering improved convenience and flexibility, leading to a higher level of compliance and superior patient management and satisfaction.

### SUMMARY OF THE INVENTION

The present invention relates to pharmaceutical compositions comprising a diphosphonic acid or a pharmaceutically acceptable salt, solvate, hydrate, prodrug, enantiomer or racemic mixture thereof. Pharmaceutical compositions of the present invention include tableting lubricants useful in the manufacture of finished pharmaceutical dosage forms. When stearic acid is used as a lubricant, the concentration is more than about 5% w/w of the total composition. The present invention further relates to compositions comprising lubricants other than stearic acid. The compositions are intended for oral administration to a person in need thereof.

In one embodiment, the invention includes the use of sodium stearyl fumarate as a lubricant.

In an embodiment, the invention includes compositions with inner and outer phases, wherein an inner phase comprises no more than about 80% w/w. In an embodiment, the invention includes an outer phase comprising at least about 20% w/w.

In one of the embodiment the present invention includes pharmaceutical compositions comprising ibandronic acid or a salt thereof wherein the drug compound has a particle size distribution: D₉₀ in the range of about 100 to 300 μm; D₅₀ in the range of about 10 to about 100 μm; and D₁₀ in the range of about 1 to about 75 μm.

In embodiments, a weight ratio of lactose to microcrystalline cellulose is from about 1:10 to about 1:1, about 1:6 to about 1:2, or about 1:5 to about 1:3. In an embodiment, a weight ratio of lactose:microcrystalline cellulose is about 1:4.

In an embodiment, the invention includes processes for preparing pharmaceutical compositions.

In an embodiment, the invention includes processes for preparing pharmaceutical compositions, which processes include a granulation step.

In another embodiment, the invention includes methods of using pharmaceutical compositions comprising ibandronic acid or salts thereof.

An embodiment of the invention provides a pharmaceutical composition comprising ibandronic acid or a salt thereof, in an amount providing about 150 mg ibandronic acid equivalent, present in an inner phase comprising no greater than about 80% by weight of the total composition, wherein an outer phase comprises at least about 20% by weight of the total composition.

Another embodiment of the invention provides a pharmaceutical composition comprising ibandronate sodium monohydrate, in an amount providing about 150 mg ibandronic acid equivalent, present in an inner phase comprising about 65% to about 70% by weight of the total composition.

A further embodiment of the invention provides a pharmaceutical composition comprising ibandronate monosodium monohydrate, in an amount providing about 150 mg ibandronic acid equivalent, present in an inner phase comprising no greater than about 80% by weight of the total composition, wherein the composition produces ibandronic acid Cₘₐₓ values about 59 ng/ml to about 94 ng/ml and ibandronic acid AUC₀₋ₜ values about 250 ng·hour/mL to about 400 ng·hour/mL, after oral administration of a single dose comprising 150 mg ibandronic acid equivalent to healthy humans.

### DETAILED DESCRIPTION OF THE INVENTION

The present invention relates to pharmaceutical compositions comprising a diphosphonic acid or a pharmaceutically acceptable salt, solvate, hydrate, prodrug, enantiomer or racemic mixture thereof. Pharmaceutical compositions of the present invention include tableting lubricants useful in the manufacture of finished pharmaceutical dosage forms. When stearic acid is used as a lubricant, its concentration is more than about 5% w/w of the total composition. The invention further relates to compositions comprising lubricants other than stearic acid. The compositions are intended for oral administration to humans in need thereof.

In an embodiment, the invention includes compositions with inner and outer phases, wherein an inner phase comprises no greater than about 80% w/w of the composition. In an embodiment, the invention includes an outer phase comprising at least about 20% w/w of the composition. In another embodiment, an inner phase comprises about 65 to about 70% w/w of the total composition.

In an embodiment, the invention includes processes for preparing pharmaceutical compositions.

In an embodiment, the invention includes processes for preparing pharmaceutical compositions, which process includes granulation.

In another embodiment, the invention includes methods of using pharmaceutical compositions comprising ibandronic acid or salts thereof.

Oral dosing of bisphosphonates typically presents significant hurdles since oral dosage forms of bisphosphonates can be corrosive to the gastrointestinal tract.

Bisphosphonates thus tend to produce adverse gastric disturbances in animals and man. The adverse gastric disturbances caused by orally dosed bisphosphonates may result in nausea, vomiting, diarrhea, bloody discharge, and ulcerations, even to the point where emergency medical intervention is required. Those bisphosphonates currently marketed to be dosed orally typically have dosing regimens, which must be closely followed by patients in order to afford minimal gastric disturbances and erosive effects. In addition the bisphosphonates, which are currently marketed, typically demonstrate low gastric absorption and resulting low bioavailability.

Furthermore it is known that ibandronic acid and ibandronate salts have shown fracture reduction efficacy with a drug free interval beyond daily administration. It is quite unexpected that fracture reduction benefits could be derived from a weekly or monthly administration of an oral biphosphonate with a single or multiple tablet administration scheme.

For purposes of the present invention, the term “inner phase” is defined as a composition comprising the active substance and at least one pharmaceutical excipient, which together constitute granules. In other words, the inner phase can be termed an intragranular composition.

For purposes of the present invention the term “outer phase” is defined as a composition optionally comprising an active substance and including at least one pharmaceutical excipient, which is combined with granules. In other words, the outer phase can be termed an extragranular composition.

In certain aspects of the present invention, a pharmaceutical composition has an inner phase comprising a granulate including active substance and one or more pharmaceutical adjuvants. The active substance according to the invention includes ibandronic acid or salts, solvates, hydrates, prodrugs, enantiomers, or racemic mixtures thereof. The proportion of active substance in the form of administration can be up to 95% by weight, relative to the total weight of the composition.

In an embodiment, the invention includes amounts of active substance in the dosage form at more than about 36% w/w.

Useful pharmaceutically acceptable salts of ibandronic acid include but are not limited to base salts of ibandronic acid such as ammonium salts, alkali metal salts such as potassium and sodium (including mono-, di-, and tri-sodium) salts, alkaline earth metal salts such as calcium and magnesium salts, salts with organic bases such as dicyclohexylamine salts, N-methyl-D-glucamine salts, and salts with amino acids such as arginine, lysine, etc. Hydrates of ibandronic acid or its salts that can be used include monohydrates, dihydrates, trihydrates, etc. For convenience, the invention will be discussed primarily with reference to the sodium salt (ibandronate monosodium monohydrate, “ibandronate sodium”), but it is not limited to use of that particular salt.

In an embodiment, a composition of the present invention includes stearic acid as a lubricant at concentrations more than about 5% w/w of the total composition.

There are instances where the rate of dissolution of a poorly soluble drug is a rate-limiting factor in its absorption by the body. It is recognized that such drugs may be more readily bioavailable if administered in a finely divided state. Because of the poor water solubility of ibandronate sodium the rate of dissolution of drug from a dosage form is a controlling factor in determining the rate and extent of drug absorption. The rate of dissolution depends on factors including particle size (or particle surface area, which can be related to particle size). Particle size also can affect how freely crystals or a powdered form of a drug will flow, which has consequences in the production processing of pharmaceutical products containing the drug.

The fractions of particles with different dimensions that exist in a powder is called the particle size distribution. It is represented in certain ways. Particle size is the maximum dimension of a particle, normally expressed in units of μm. Particle size distributions can be expressed in terms of, D₁₀, D₅₀, D₉₀ and D_{[4,3]}. The D₁₀, D₅₀ and D₉₀ represent the 10th, the median or 50th percentile, and the 90th percentile of the particle size distribution, respectively, as measured by volume. That is, the D₁₀, D₅₀, D₉₀ is a value of the distribution such that 10%, 50%, 90% by volume of the particles have a size of this value or less, or is the percentage of particles smaller than that size. D₅₀ is also known as median diameter of particle. It is one of the important parameters representing characteristics of particle of powder. For a sample, if D₅₀=5 μm, it means that 50% of the particles are smaller than 5 μm. Similarly, if D₁₀=5 μm, 10% by volume of the particles are less than or equal to 5 μm, and if D₉₀=5 μm, 90% of the particles are less than or equal to 5 μm. D_{[4,3]} is the volume moment mean of the particle, or the volume weighted particle size.

In an embodiment the present invention includes pharmaceutical compositions comprising ibandronic acid or its salts wherein D₉₀ is in the range of about 100 to 300 μm, D₅₀ is in the range of about 10 to about 100 μm, and D₁₀ is in the range of about 1 to about 75 μm.

In embodiments, a ratio of D₁₀ to D₉₀ for ibandronic acid or a salt thereof ranges from about 1:1 to about 1:25. In certain embodiments, a ratio of D₁₀ to D₉₀ for ibandronic acid or a salt thereof ranges from about 1:10 to about 1:15.

In an embodiment, a composition of the present invention includes a lubricant other than stearic acid.

The selection and proportion of a suitable lubricant in an outer phase is particularly important, since it influences the physical properties of a dosage form. The choice and proportion of lubricant determine whether the substance filling the capsule or being formed into a tablet can be processed without difficulty on equipment over a prolonged period, including whether the tablets stick to compression moulding dies in a tableting machine.

Sufficient lubricant will typically therefore be added to an outer phase. If, however, the proportion of lubricant is too high, there may be other adverse effects. For example a granulate may become so water-repellent that a resulting tablet disintegrates very slowly and the desired drug dissolution rate is not reached. The lubricant selected primarily should be compatible with active substance and any other components.

Various useful lubricants for the present invention include, but are not limited to, stearic acid derivatives such as magnesium stearate, sodium stearyl fumarate, calcium stearate, etc., talc, macrogol (polyethylene glycol), hydrogenated esters of fatty acids with glycerine, glyceryl behenate, hydrogenated castor oil, and mixtures thereof.

In another embodiment the invention includes use of sodium stearyl fumarate, magnesium stearate, or glyceryl behenate as a lubricant.

In one embodiment, the invention includes sodium stearyl fumarate as a lubricant.

In another embodiment, the invention includes more than about 5% w/w of stearic acid as a lubricant.

In another embodiment, the invention includes a concentration of lubricant from about 0.1% w/w to about 10% w/w.

In another embodiment of the invention, compositions comprising lubricants other than stearic acid are formed into tablets using a direct compression process.

In yet another embodiment of the present invention, pharmaceutical compositions comprising a bisphosphonic acid or pharmaceutically acceptable salts, solvates, hydrates, prodrugs, enantiomers or racemic mixtures thereof are substantially devoid of lubricants.

The pharmaceutical compositions of the present invention may also contain one or more additional formulation ingredients or excipients from a wide variety of excipients known in the pharmaceutical formulation art. According to the desired properties of the formulation, any number of ingredients may be selected, alone or in combination, based upon their uses in preparing compositions. Such ingredients include, but are not limited to, diluents, disintegrants, binders, glidants, lubricants (discussed above), solvents, flavours, colours, sweetners, and preservatives.

### Diluents:

Various useful diluents include but are not limited to starches, lactose, mannitol, cellulose derivatives and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

In an embodiment the present invention includes pharmaceutical compositions wherein the diluents used are lactose, microcrystalline cellulose or combinations thereof.

In embodiments, a weight ratio of lactose to microcrystalline cellulose is from about 1:10 to about 1:1, about 1:6 to about 1:2, or about 1:5 to about 1:3. In an embodiment, a weight ratio of lactose:microcrystalline cellulose is about 1:4. Varying the ratio can cause difficulties in tableting.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not being limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcellulose. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

### Binders:

Various useful binders include but are not limited to hydroxypropylcellulose (Klucel™ LF), hydroxypropyl methylcellulose (Methocel™), polyvinylpyrrolidone or povidone (PVP-K25, PVP-K29, PVP-K30), powdered acacia, gelatin, guar gum, carbomer (carbopol), methylcellulose, polymethacrylates, and starch.

### Glidants:

Various useful glidants or antisticking agents include but are not limited to talc, silica derivatives, colloidal silicon dioxide.

### Solvents:

Various solvents that are useful in processing include but are not limited to water, lower alcohols like methanol, ethanol, and isopropanol, acidified ethanol, acetone, polyols, polyethers, oils, esters, alkyl ketones, methylene chloride, castor oil, ethylene glycol monoethyl ether, diethylene glycol monobutyl ether, diethylene glycol monoethyl ether, dimethylsulphoxide, dimethylformamide, and tetrahydrofuran.

### Flavors:

The flavoring agents, which can be used in the present invention, include but are not limited to natural or synthetic or semi-synthetic flavors like menthol, fruit flavors, citrus oils, peppermint oil, spearmint oil, oil of wintergreen (methyl salicylate), etc.

### Colorants:

Various useful colorants include but are not limited to Food Yellow No. 5, Food Red No. 2, Food Blue No. 2, and the like, food lake colorants, and ferric oxide.

### Preservatives:

Preservatives can desirably be incorporated into pharmaceutical products for stabilizing against the growth of potentially harmful microorganisms. While microorganisms tend to grow in an aqueous environment, microorganisms can also reside in a hydrophobic or oil phase. Suitable preservatives for compositions of the present invention include alkyl esters of para-hydroxybenzoic acid like methylparaben and propylparaben, benzoates, hydantoin derivatives, propionate salts, sorbic acid, benzyl alcohol, imidazolidinyl urea, sodium dehydroacetate and a variety of quaternary ammonium compounds. Appropriate preservatives can be employed to satisfy a preservative challenge test and to provide product stability. The preservative can be chosen based on the consideration of possible incompatibilities between the preservative and other ingredients in the release system. Preservatives can be employed in amounts ranging from about 0.01% to about 2% by weight of the composition.

As discussed above, it is known that diphosphonic acids, including ibandronic acid or its salts give rise to irritations of the upper gastrointestinal tract. In order to solve these problems, orally available administration forms frequently are coated. Film forming polymers are frequently used for coating.

### Film-Forming Agents:

Various film-forming agents include but are not limited to cellulose derivatives such as soluble alkyl- or hydroalkyl-cellulose derivatives such as methyl cellulose, hydroxymethyl cellulose, hydroxyethyl cellulose, hydroxypropyl cellulose, hydroxymethyethyl cellulose, hydroxypropyl methylcellulose, sodium carboxymethyl cellulose, etc., acidic cellulose derivatives such as cellulose acetate phthalate, cellulose acetate trimellitate and methylhydroxypropylcellulose phthalate, polyvinyl acetate phthalate, etc., insoluble cellulose derivatives such as ethylcellulose and the like, dextrins, starches and starch derivatives, polymers based on carbohydrates and derivatives thereof, natural gums such as gum Arabic, xanthans, alginates, polyacrylic acid, polyvinylalcohol, polyvinyl acetate, polyvinylpyrrolidone, polymethacrylates and derivatives thereof (Eudragit™), chitosan and derivatives thereof, shellac and derivatives thereof, and waxes and fat substances.

In the case of polymethacrylates, cationic copolymerizates of dimethylaminoethyl methacrylate with neutral methacrylic esters (Eudragit™ E), copolymerizates of acrylic and methacrylic esters having a low content of quaternary ammonium groups (described in “Ammonio Methacrylate Copolymer Type A or Type B” USP/NF, Eudragit™ RL and RS, respectively), and copolymerizates of ethyl acrylate and methyl methacrylate with neutral character (in the form of an aqueous dispersion, described in “Polyacrylate Dispersion 30 Per Cent” Ph. Eur., Eudragit™ NE 30 D) are useful.

Anionic copolymerizates of methacrylic acid and methyl methacrylate (described in “Methacrylic Acid Copolymer, Type C” USP/NF, Eudragit™ L and S, respectively, or in the form of the Eudragit™ L 30 D aqueous dispersion), acidic cellulose derivatives such as cellulose acetate phthalate, cellulose acetate trimellitate and methylhydroxypropylcellulose phthalate, polyvinyl acetate phthalate, etc. may be used for film coatings.

The coating may be applied using methods such as film coating, press coating, tablet coating, encapsulating or microencapsulating.

If required, the films may contain additional adjuvants for coating processing such as plasticizers, polishing agents, colorants, pigments, antifoam agents, opacifiers, antisticking agents, and the like.

### Plasticizers:

Various plasticizers include but are not limited to castor oil, diacetylated monoglycerides, dibutyl sebacate, diethyl phthalate, glycerin, polyethylene glycol, propylene glycol, triacetin, and triethyl citrate. Also mixtures of plasticizers may be utilized. The type of plasticizer depends upon the type of coating agent. A plasticizer is frequently present in an amount ranging from 5% (w/w) to 30% (w/w), based on the total weight of the film coating.

An opacifier like titianium dioxide may also be present in an amount ranging from about 10% (w/w) to about 20% (w/w), based on the total weight of the coating. When coloured tablets are desired then the colour is normally applied in the coating. Consequently, colouring agents and pigments may be present in the film coating. Various colouring agents include but are not limited to ferric oxides, which can be red, yellow, black or blends thereof.

Anti-adhesives are normally used in film coating processes to avoid sticking effects during film formation and drying. An example of an anti-adhesive for this purpose is talc. The anti-adhesive can be present in the film coating in an amount of about 5% (w/w) to 15% (w/w), based upon the total weight of the coating.

Suitable polishing agents include polyethylene glycols of various molecular weights or mixtures thereof, talc, surfactants (e.g. glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax). In an embodiment, polyethylene glycols having molecular weights of 3,000-20,000 are employed.

In addition to above the coating ingredients, sometimes ready-mixed coating materials such as Opadry™ White OY 58900 (contains hydroxypropyl methyl cellulose, PEG 6000, and titanium dioxide), Lusterclear™, etc. will be used. Lustreclear is a combination of microcrystalline cellulose and carrageenan. Lustreclear is used as an aqueous clear film coating. It allows for a short hydration time prior to coating and fast drying. Its smooth satin-like finish eliminates edge wear and logo bridging, thus Lustreclear provides a distinctive smooth finish, which enables easy swallowing due to its smooth surface. The commercially available products are either provided as dispersions in a liquid, or are solids that can be dispersed in a suitable liquid for use.

An embodiment of the invention includes pharmaceutical preparations made in accordance with the invention that are solid dosage forms, which include but are not limited to capsules, tablets, caplets, pills, powders, granules, etc.

In an embodiment, the invention includes the use of packaging materials such as containers and lids of high-density polyethylene (HDPE), low-density polyethylene (LDPE) and or polypropylene and/or glass, and blisters or strips composed of aluminium or high-density polypropylene, polyvinyl chloride, polyvinylidene dichloride, etc.

### Processes for Preparing the Compositions:

The pharmaceutical compositions may be prepared using different formulation techniques such as physical mixing, blending, wet granulation, dry granulation, direct compression, fluid bed granulation, etc., and combinations thereof.

In an embodiment of the invention, direct compression is used to avoid contact with water.

An aspect of the present invention is further directed to processes for preparing pharmaceutical compositions comprising ibandronic acid or salts thereof, wherein an embodiment of a process comprises:

a) Sifting drug, diluents, disintegrants through a sieve.

b) Dry mixing sifted drug, diluents, and disintegrants.

c) Granulating the dry mix with a binder solution.

d) Drying the granules.

e) Passing the dried granules through a sieve.

f) Mixing the dried granules with sifted extragranular materials and blending.

g) Compressing the blend into tablets.

h) Coating the tablets with a coating dispersion.

Tablets can be subjected to an in vitro dissolution evaluation according to Test 711 “Dissolution” in United States Pharmacopeia 24, United States Pharmacopeial Convention, Inc, Rockville, Md., 1999 (“USP”) to determine the rate at which the active substance is released from the dosage forms, and the content of active substance can be determined in solutions using techniques such as high performance liquid chromatography. The pharmaceutical dosage forms of the present invention are intended for oral administration to a patient in need thereof.

In certain embodiments of the invention, tablets prepared according to the invention and containing 150 mg of ibandronic acid equivalent provide, after oral administration of a single dose to healthy humans, maximum plasma concentrations (Cₘₐₓ) about 59 to about 94 ng/mL. In certain embodiments of the invention, tablets prepared according to the invention and containing 150 mg of ibandronic acid equivalent provide, after oral administration of a single dose to healthy humans, areas under the plasma concentration vs. time curve (AUC₀₋ₜ) about 250 to about 400 ng·hour/mL.

Certain specific aspects and embodiments of the invention will be further described in the following examples, which are provided only for purposes of illustration and are not intended to limit the scope of the invention in any manner.

### EXAMPLE 1

### Ibandronic Acid 150 mg Tablets

| Ingredient | mg/Tablet |
|---|---|
| Ibandronate sodium | 168.75 |
| Lactose monohydrate | 18.75 |
| Microcrystalline cellulose | 105 |
| (Avicel ™ PH 101) | |
| Crospovidone | 13 |
| Povidone K 30 | 18.75 |
| Water ‡ | 215.625 |
| Microcrystalline cellulose (Avicel | 22.45 |
| PH 101) | |
| Crospovidone | 13.3 |
| Colloidal silicon dioxide | 7.5 |
| Sodium stearyl fumarate | 7.5 |
| Total | 375 |

Coating

| | |
|---|---|
| Opadry ™ White OY 58900 | 11.25 |
| Water ‡ | 101.25 |

‡ Evaporates during processing.

Opadry White OY 58900 contains hydroxypropyl methylcellulose, PEG 6000, and titanium dioxide.

### Manufacturing Process:

a) Sifted drug, microcrystalline cellulose, lactose monohydrate, and crospovidone through a #40 mesh sieve.

b) The sifted materials from step a) were dry mixed in a rapid mixer granulator for about 10 minutes

c) Povidone K 30 was mixed with water and kept aside until it formed a clear solution.

d) The dry mixed materials in step b) were granulated using binder solution prepared in step c).

e) The granules were dried in a fluid bed drier at 60° C.±5° C. until loss on drying was below 2% w/w.

f) The dried granules were sifted through a #24 mesh sieve.

g) The extragranular materials microcrystalline cellulose, crospovidone, and colloidal silicon dioxide were sifted through a #40 mesh sieve.

h) Mixed the dried granules from step f) with sifted extragranular materials from step g) and blended for about 25 minutes.

i) Sifted sodium stearyl fumarate through a #80 mesh sieve.

j) Added sifted sodium stearyl fumarate to the blend of step h) and blended for about 5 minutes.

k) Compressed the above-prepared blend into tablets using a tablet compression machine.

l) Dispersed Opadry in water and stirred about 45 minutes to get a homogenous dispersion.

m) Coated the tablets with Opadry suspension in a perforated coating pan with an inlet temperature of 60° C.±5° C. to get a weight buildup of 3% w/w.

### EXAMPLE 2

### Ibandronic Acid 150 mg Tablets (Magnesium Stearate as Lubricant)

| Ingredient | mg/Tablet |
|---|---|
| Ibandronate sodium | 168.75 |
| Lactose monohydrate | 18.75 |
| Microcrystalline cellulose (Avicel PH 101) | 105 |
| Crospovidone | 13 |
| Povidone K 30 | 18.75 |
| Water ‡ | 215.625 |
| Microcrystalline cellulose (Avicel PH 101) | 22.45 |
| Crospovidone | 13.3 |
| Colloidal silicon dioxide | 7.5 |
| Magnesium stearate | 7.5 |

Coating

| | |
|---|---|
| Opadry White OY 58900 | 11.25 |
| Water ‡ | 101.25 |

‡ Evaporates during processing.

Manufacturing process: same as that of Example 1.

### EXAMPLE 3

### Ibandronic Acid 150 mg Tablets (Stearic Acid More than 5% as a Lubricant)

| Ingredient | mg/Tablet |
|---|---|
| Ibandronate sodium | 168.75 |
| Lactose monohydrate | 18.75 |
| Microcrystalline cellulose (Avicel PH 101) | 86.3 |
| Crospovidone | 13 |
| Povidone K 30 | 18.75 |
| Water ‡ | 215.625 |
| Microcrystalline cellulose (Avicel PH 101) | 22.45 |
| Crospovidone | 13.3 |
| Colloidal silicon dioxide | 7.5 |
| Stearic acid | 26.25 |

Coating

| | |
|---|---|
| Opadry White OY 58900 | 11.25 |
| Water ‡ | 101.25 |

‡ Evaporates during processing.

Manufacturing process: same as that of Example 1.

### EXAMPLE 4

### Ibandronic Acid 150 mg Tablets (Hydrogenated Castor Oil as a Lubricant)

| Ingredient | mg/Tablet |
|---|---|
| Ibandronate sodium | 168.75 |
| Microcrystalline cellulose (Avicel PH 101) | 193.5 |
| Crospovidone | 9 |
| Povidone K 30 | 11.25 |
| Water ‡ | 101.25 |
| Microcrystalline cellulose (Avicel PH 112) | 22.5 |
| Crospovidone | 22.5 |
| Colloidal silicon dioxide | 9 |
| Hydrogenated castor oil | 13.5 |

Coating

| | |
|---|---|
| Lustreclear* | 13.5 |
| Water ‡ | 121.5 |

‡ Evaporates during processing.
*Lustreclear ™ is a microcrystalline cellulose and carrageenan based coating material and is supplied by FMC Biopolymer.

Manufacturing process: same as that of Example 1, with substitution of Lustreclear for Opadry.

### EXAMPLE 5

### Ibandronic Acid 150 mg Tablets (Glyceryl Behenate as a Lubricant)

| Ingredient | mg/Tablet |
|---|---|
| Ibandronate sodium | 168.75 |
| Microcrystalline cellulose (Avicel PH 101) | 193.5 |
| Crospovidone | 9 |
| Povidone K 30 | 11.25 |
| Water ‡ | 101.25 |
| Microcrystalline cellulose (Avicel PH 112) | 22.5 |
| Crospovidone | 22.5 |
| Colloidal silicon dioxide | 9 |
| Glyceryl behenate | 13.5 |

Coating

| | |
|---|---|
| Lustreclear ™ | 13.5 |
| Water ‡ | 121.5 |

‡ Evaporates during processing.

Manufacturing process: same as that of Example 4.

The coated tablets prepared above were subjected to dissolution testing with the following dissolution conditions:

Medium: 500 ml purified water

RPM: 50

Apparatus: USP II paddle type and the results obtained after 15 and 30 minutes are shown in Table 1.

TABLE 1

| Time (minutes) | Cumulative % Drug Dissolved |
|---|---|
| 15 | 103 |
| 30 | 115 |

### EXAMPLE 6

### Ibandronic Acid 150 mg Tablets (Magnesium Stearate as a Lubricant)

| Ingredient | mg/Tablet |
|---|---|
| Ibandronate sodium | 168.75 |
| Microcrystalline cellulose (Avicel PH 101) | 193.5 |
| Crospovidone | 9 |
| Povidone K 30 | 11.25 |
| Water ‡ | 101.25 |
| Microcrystalline cellulose (Avicel PH 112) | 22.5 |
| Crospovidone | 22.5 |
| Colloidal silicon dioxide | 9 |
| Magnesium stearate | 13.5 |

Coating

| | |
|---|---|
| Lustreclear | 13.5 |
| Water ‡ | 121.5 |

‡ Evaporates during processing.

Manufacturing process: same as that of Example 4.

The coated tablets prepared above were subjected to dissolution testing, with the following dissolution conditions:

Medium: 500 ml purified water

RPM: 50

Apparatus: USP II paddle type

and the results obtained after 15 and 30 minutes are shown in Table 2.

TABLE 2

| Time (minutes) | Cumulative % Drug Dissolved |
|---|---|
| 15 | 107 |
| 30 | 112 |

### EXAMPLE 7

### Ibandronic Acid 150 mg Tablets (Sodium Stearyl Fumarate as a Lubricant)

| Ingredient | mg/Tablet |
|---|---|
| Ibandronate sodium | 168.75 |
| Lactose monohydrate | 18.75 |
| Microcrystalline cellulose (Avicel PH 101) | 75 |
| Povidone K 30 | 18.75 |
| Water ‡ | 215.625 |
| Microcrystalline cellulose (Avicel PH 102) | 60 |
| Crospovidone | 26.25 |
| Colloidal silicon dioxide | 3.75 |
| Sodium stearyl fumarate | 3.75 |

Coating

| | |
|---|---|
| Lustreclear | 11.25 |
| Water ‡ | 101.25 |

‡ Evaporates during processing.

Manufacturing process: same as that of Example 4.

The coated tablets prepared as above were subjected to dissolution testing with the following dissolution conditions:

Medium: 500 ml purified water

RPM: 50

Apparatus: USP II paddle type

and the results obtained after 15 and 30 minutes are shown in Table 3.

TABLE 3

| Time (minutes) | Cumulative % Drug Dissolved |
|---|---|
| 15 | 89 |
| 30 | 90 |

### EXAMPLE 8

### Ibandronic Acid 150 mg Tablets (PEG 6000 as a Lubricant)

| Ingredient | mg/Tablet |
|---|---|
| Ibandronate sodium | 168.75 |
| Lactose monohydrate | 85 |
| Microcrystalline cellulose (Avicel PH 101) | 90.5 |
| Crospovidone | 9 |
| Povidone K 30 | 11.25 |
| Isopropyl alcohol ‡ | 101.25 |
| Microcrystalline cellulose (Avicel PH 102) | 45 |
| Crospovidone | 22.5 |
| Colloidal silicon dioxide | 9 |
| PEG 6000 | 9 |

Coating

| | |
|---|---|
| Lustreclear | 13.5 |
| Water ‡ | 121.5 |

‡ Evaporates during processing.

Manufacturing process: same as that of Example 4.

### EXAMPLE 9

### Ibandronic Acid 150 mg Tablets

| Ingredient | mg/Tablet |
|---|---|
| Ibandronate sodium | 168.75 |
| Lactose monohydrate | 18.75 |
| Microcrystalline cellulose (Avicel PH 101) | 75 |
| Povidone K 30 | 18.75 |
| Water ‡ | 75 |
| Microcrystalline cellulose (PH 102) | 60 |
| Crospovidone XL 10 | 26.25 |
| Colloidal silicon dioxide | 3.75 |
| Sodium stearyl fumarate | 3.75 |

Coating

| | |
|---|---|
| Opadry White OY58900** | 11.25 |
| Water ‡ | 101.25 |

‡ Evaporates during processing.
**OPADRY White OY58900 contains HPMC 2910/hypromellose 5 cP, titanium dioxide, and macrogol/PEG 400; supplied by Colorcon.

Manufacturing Process:

a) Ibandronate sodium, microcrystalline cellulose, and lactose monohydrate were sifted through a #30 mesh sieve.

b) The sifted materials from step a) were dry mixed in a rapid mixer granulator for about 15 minutes with impeller at fast speed and chopper off.

c) Povidone K 30 was mixed with water with continuous stirring and kept aside until it formed a clear solution.

d) The dry mixed materials in step b) were granulated using binder solution prepared in step c).

e) The granules were dried in a fluid bed dryer at 60° C.±5° C. until loss on drying was 1-2% w/w.

f) The dried granules were sifted through a #24 mesh sieve.

g) The extragranular materials microcrystalline cellulose, crospovidone, and colloidal silicon dioxide were sifted through a #40 mesh sieve.

h) The dried granules from step f) were mixed with sifted extragranular material from step g) and blended in a double cone blender for about 25 minutes.

i) Sodium stearyl fumarate was sifted through a #60 mesh sieve.

j) The sodium stearyl fumarate from step i) was added to the blend of step h) and blended for about 10 minutes.

k) The above-prepared blend was compressed into tablets using a tablet compression machine.

l) Opadry White was dispersed in water with continuous stirring for about 30 minutes to get a homogenous dispersion.

m) Coated the tablets with Opadry suspension in a perforated coating pan with an inlet temperature of 65° C.±5° C. to get a weight buildup of 2.5±0.5% w/w.

The coated tablets prepared as above were subjected to dissolution testing, using the following dissolution conditions:

Medium: 500 ml purified water

RPM: 50

Apparatus: USP 11 paddle type.

The results are shown in Table 4.

TABLE 4

| Time (minutes) | Cumulative % Drug Dissolved |
|---|---|
| 5 | 62 |
| 10 | 84 |
| 15 | 89 |
| 30 | 94 |

The coated tablets were used in a randomized, two-way crossover, open-label, single-dose, fasting state clinical study with a washout period of at least four weeks between dosing. The study involved administration of single doses of the test product and, as a reference, the commercial product BONIVA, to 76 healthy human volunteers, and plasma ibandronic acid concentrations were determined at intervals after dosing.

The following parameters were calculated:

AUC₀₋ₜ=the area under plasma concentration versus time curve, from time zero following administration to the last measurable concentration, expressed in ng·hour/mL units.

Cₘₐₓ=maximum plasma concentration following administration, expressed in ng/mL units.

The results of these pharmacokinetic parameters in the study were calculated and are summarized Table 5.

TABLE 5

| | AUC₀₋ₜ | | Cₘₐₓ | |
|---|---|---|---|---|
| Parameter | Test | Reference | Test | Reference |
| Least Square Mean log | 5.8146 | 5.7580 | 4.3838 | 4.3111 |
| Geometric Mean | 335.18 | 316.72 | 80.14 | 74.53 |

| | | |
|---|---|---|
| 100 × (Test ÷ Reference | 106 | 108 |
| Geometric Mean Values) | | |

### EXAMPLE 10

### Ibandronic Acid 150 mg Tablets (7:22 Weight Ratio of Lactose:Microcrystalline Cellulose in the Inner Phase)

| Ingredient | mg/Tablet |
|---|---|
| Ibandronate sodium | 168.75 |
| Lactose monohydrate | 26.25 |
| Microcrystalline cellulose (Avicel PH101) | 82.5 |
| PVP K30 | 18.75 |
| Water ‡ | 75 |
| Microcrystalline cellulose (Avicel PH102) | 60 |
| Crospovidone XL 10 | 26.25 |
| Colloidal silicon dioxide (Aerosil ™ 200) | 3.75 |
| Sodium stearyl fumarate | 3.75 |

Coating

| | |
|---|---|
| Opadry White OY58900 | 11.25 |
| Water ‡ | 101.25 |

‡ Evaporates during processing.

Manufacturing process: same as that of Example 9.

### EXAMPLE 11

### Ibandronic Acid 150 mg Tablets (1:6 Weight Ratio of Lactose:Microcrystalline Cellulose in the Inner Phase)

| Ingredient | mg/Tablet |
|---|---|
| Ibandronate sodium | 168.75 |
| Lactose monohydrate | 11.25 |
| Microcrystalline cellulose (Avicel PH101) | 67.5 |
| PVP K30 | 18.75 |
| Water ‡ | 75 |
| Microcrystalline cellulose | 75 |
| (Avicel PH102) | |
| Crospovidone XL 10 | 26.25 |
| Colloidal silicon dioxide (Aerosil 200) | 3.75 |
| Sodium stearyl fumarate | 3.75 |

Coating

| | |
|---|---|
| Opadry White OY58900 | 11.25 |
| Water ‡ | 101.25 |

‡ Evaporates during processing.

Manufacturing process: same as that of Example 9.

We claim:

1. A pharmaceutical composition comprising ibandronic acid or a salt thereof, in an amount providing about 150 mg ibandronic acid equivalent, present in an inner phase comprising no greater than about 80% by weight of the total composition, wherein an outer phase comprises at least about 20% by weight of the total composition.

2. The pharmaceutical composition of claim 1, wherein the composition produces ibandronic acid Cₘₐₓ values about 59 ng/ml to about 94 ng/ml, after oral administration of a single dose comprising 150 mg ibandronic acid equivalent to healthy humans.

3. The pharmaceutical composition of claim 1, wherein the composition produces ibandronic acid AUC₀₋ₜ values about 250 ng·hour/mL to about 400 ng·hour/mL, after oral administration of a single dose comprising 150 mg ibandronic acid equivalent to healthy humans.

4. The pharmaceutical composition of claim 1, comprising a lactose:microcrystalline cellulose weight ratio in the inner phase about 1:6 to about 1:2.

5. The pharmaceutical composition of claim 1, comprising a lactose:microcrystalline cellulose weight ratio in the inner phase about 1:4.

6. The pharmaceutical composition of claim 1, wherein a ratio of D₁₀ to D₉₀ for ibandronic acid or a salt thereof ranges from about 1:1 to about 1:25.

7. The pharmaceutical composition of claim 1, wherein a ratio of D₁₀ to D₉₀ for ibandronic acid or a salt thereof ranges from about 1:10 to about 1:15.

8. The pharmaceutical composition of claim 1, wherein ibandronic acid or a salt thereof comprises ibandronate monosodium monohydrate.

9. A pharmaceutical composition comprising ibandronate sodium monohydrate, in an amount providing about 150 mg ibandronic acid equivalent, present in an inner phase comprising about 65% to about 70% by weight of the total composition.

10. The pharmaceutical composition of claim 9, wherein an inner phase comprises a lactose:microcrystalline cellulose weight ratio about 1:6 to about 1:2.

11. The pharmaceutical composition of claim 9, wherein an inner phase comprises a lactose:microcrystalline cellulose weight ratio about 1:4.

12. The pharmaceutical composition of claim 9, wherein the composition produces ibandronic acid Cₘₐₓ values about 59 ng/ml to about 94 ng/ml after oral administration of a single dose comprising 150 mg ibandronic acid equivalent, to healthy humans.

13. The pharmaceutical composition of claim 9, wherein the composition produces ibandronic acid AUC₀₋ₜ values about 250 ng·hour/mL to about 400 ng·hour/mL, after oral administration of a single dose comprising 150 mg ibandronic acid equivalent to healthy humans.

14. The pharmaceutical composition of claim 9, wherein a ratio of D₁₀ to D₉₀ for ibandronate monosodium monohydrate ranges from about 1:1 to about 1:25.

15. The pharmaceutical composition of claim 9, wherein a ratio of D₁₀ to D₉₀ for ibandronate monosodium monohydrate ranges from about 1:10 to about 1:15.

16. A pharmaceutical composition comprising ibandronate monosodium monohydrate, in an amount providing about 150 mg ibandronic acid equivalent, present in an inner phase comprising no greater than about 80% by weight of the total composition, wherein the composition produces ibandronic acid Cₘₐₓ values about 59 ng/ml to about 94 ng/ml and ibandronic acid AUC₀₋ₜ values about 250 ng·hour/mL to about 400 ng·hour/mL, after oral administration of a single dose comprising 150 mg ibandronic acid equivalent to healthy humans.

17. The pharmaceutical composition of claim 16, wherein an inner phase comprises a lactose:microcrystalline cellulose weight ratio about 1:6 to about 1:2.

18. The pharmaceutical composition of claim 16, wherein an inner phase comprises a lactose:microcrystalline cellulose weight ratio about 1:4.

19. The pharmaceutical composition of claim 16, wherein a ratio of D₁₀ to D₉₀ for ibandronate monosodium monohydrate ranges from about 1:1 to about 1:25.

20. The pharmaceutical composition of claim 16, wherein a ratio of D₁₀ to D₉₀ for ibandronate monosodium monohydrate ranges from about 1:10 to about 1:15.
